(19) United States
(12) Patent Application Publication
(10) Pub. No.: US 2025/0111937 A1
(43) Pub. Date: Apr. 3, 2025

(54) SUBJECT-CENTRIC SMART HOSPITAL WITH CUSTOMIZABLE SMART SERVICES

(71) Applicant: Cerner Innovation, Inc., Kansas City, MO (US)

(72) Inventors: Praveen Bhat Gurpur, Bengaluru (IN); Ranjani Rajagopalan, Coimbatore (IN); Priyanka Verma, London (GB); Suchitra Joyce Phillips, Bangalore (IN); Kishore Kumar Naik Pujari, Bengaluru (IN); Ankur Chatter, Rajasthan (IN); Amarrtya Jana, Kolkata (IN); Harish Raghupatruni, Bangalore (IN); Praveen Kumar Patil, Bangalore (IN)

(73) Assignee: Cerner Innovation, Inc., Kansas City, MO (US)

(21) Appl. No.: 18/764,992

(22) Filed: Jul. 5, 2024

Related U.S. Application Data
(60) Provisional application No. 63/586,679, filed on Sep. 29, 2023.

(51) Int. Cl. G16H 40/20; G06F 3/01; G16H 10/60; G16H 50/20

(52) U.S. Cl. CPC G16H 40/20; G06F 3/011; G16H 10/60; G16H 50/20

(57) ABSTRACT

Techniques disclosed herein provide a layered healthcare medical stack for embodying the architecture of a subject-centric smart hospital (SH) with customizable smart services. A subject centric matrix hosted on a centralized cloud that is configured to provide interoperability, integration, and customization of services for various healthcare organizations. Data is obtained for each subject to create a subject-specific persona for performing prescriptive and predictive analysis thereby generating alerts and trends based on the current and historical healthcare data of a subject. The subject-centric matrix can enable healthcare facilities to choose any service in any layer without a prerequisite service to be in place first. The healthcare medical stack enables dynamically changing services to the subjects as their health profile and status changes such that they get a personalized and customized experience that is created based on the current health status, which is different from the one observed in the previous visit.

### CROSS-REFERENCE TO RELATED APPLICATIONS

This application claims the benefit of and the priority to U.S. Provisional Patent Application 63/586,679, filed on Sep. 29, 2023, which is hereby incorporated by reference in its entirety for all purposes.

### BACKGROUND

Hospitals have different medical information systems for managing various workflows, which currently operate in silos that results in suboptimal and inefficient hospital management as a unified and emerging operational picture of the hospital are not visible to decision makers and stakeholders in real time. This can result, not only in a loss of operational efficiency thereby significantly increasing the cost of providing healthcare services, but also in a frustrating experience for their patients. The inability of these systems to deliver timely access to subject information frequently impedes medical decision-making and may even endanger the safety of patients. Patients also frequently feel disconnected with the healthcare experiences and lack information regarding their medical histories and treatment plans. These challenges have been somewhat addressed by health cloud platforms and applying data-driven analytics to provide an evidence-based experience. While these technologies are included in one or the other form in existing smart hospital systems, a subject-centric system that provides the healthcare delivery, customizes the smart services based on preferences of a subject or a healthcare facility and personalizes the experience of a subject in response to the evolving health profile of a subject may be needed in the health ecosystems.

Early versions of intelligent healthcare systems were focused on resource management and automating administrative and billing services. While these innovations helped to run hospital operations efficiently, the immediate impact on subject centric experiences may be frequently overlooked. Despite collecting feedback from subjects, hospital management struggles to have access to the systems to objectively analyze the feedback and identify areas of concern that need its immediate attention. Additionally, the seamless view of the health of a subject for meticulous diagnoses and personalized care regimens is still a distant reality because of the difficulty of integrating the data of subjects across different departments and specialties.

Effective and secure real-time digital communication between patients and hospital staff promote enhanced patient satisfaction, patient engagement, and reduced hospital readmissions. Therefore, a subject centric matrix that motivates technology adoption, secure HIPAA compliant communication exchange channel between providers and subjects, integrated health solutions, descriptive and social analysis of individuals for creating individual personas for provision of customized services, thus leading to a structured yet interactive health facility that provides a happy and interactive experience to subjects at a reduced cost.

### SUMMARY

Certain aspects and features of the present disclosure relate to a subject-centric smart hospital for providing customized healthcare facilities. The system includes a subject-centric medical stack, including an application layer, a services layer, and a centralized cloud platform. The cloud platform further includes an interoperability module configured to synchronize disparate datasets that support data exchange between multiple organizations or departments. An integration module that provides consistent data format and exchange of data between various services. A customization module that provides customization of services to accommodate the needs and preferences of individual subjects or healthcare facilities.

In some embodiments, a computer-implemented method is provided that includes a subject-centric medical stack, including an application layer that is configured to collect data in real-time from at least one data source for one or more subjects where a data source includes a subject-mounted device, a network device, an administration application, a clinical application, or a subject application. The collected data can be transformed to establish a secure communication channel between the data sources through an integration of interfaces. The transformed data, collected in real-time from multiple sources, can be aggregated with the data that is retrieved from a number of electronic health records of the associated subjects to build a subject persona. Further, a services layer is linked with the application layer through the application programming interfaces (API), where the service layer includes a subject-centric matrix that includes sublayers representing services to be deployed. Each sublayer can be customized for a given client (e.g., hospital) or even for a particular subject. The services represented in the sublayers can include a smart service that uses a data processing module to collect and store data, a communication interface module for data exchange, and an artificial intelligence-based engine to perform data analytics on the collected data to enable smart inferencing.

In some embodiments, the subject-centric matrix includes a first sublayer including services configured to know the subject better (that is, to gain information about the subject), which may include integrating multiple healthcare records, processing sentiment analysis of feedback of a subject to create the subject-specific health persona, and/or providing a real-time communication for engagement among a number of subjects. A second sublayer can include services, targeted for collecting data from subject-based applications and wearable analytics for predictive and prescriptive analysis of subjects within and outside a healthcare facility to preferably provide a preventive approach rather than a curative one. A third sublayer can include advanced services based on data-driven and artificial intelligence (AI) to enhance subject experience such as for quick registration, augmented/virtual reality (AR/VR), voice scribe, a smart building with internet of thing (IoT) devices, and real-time tracking of subjects.

The services can be hosted on a centralized cloud platform comprising of an interoperability module configured to synchronize disparate datasets that support a data exchange between a multiple organizations or departments. An integration module can provide consistent data format and exchange of data between various services. A customization module can provide customization of services to accommodate the needs and preferences of individual subjects or healthcare facilities. The cloud platform further can provide secure and scalable infrastructure for storing, processing, and accessing healthcare data and applications. Furthermore, wearable devices and AI driven techniques can be used to obtain the real-time data of a subject, such as biometrics and/or the updated medical test reports, and such data can be stored into the electronic medical record (EMR) for providing further assistance. The data obtained may be used to create a subject persona and for performing prescriptive and predictive analysis, thereby generating alerts and trends based on the current and historical health data of a subject. The subject-centric matrix can be built to enable healthcare facilities to choose any service in any layer without a prerequisite service to be in the place first.

The subject-centric smart-hospital system of the present disclosure may provide a computer-implemented method that integrates outputs of subject-mounted devices to support remote monitoring, dynamic prediction of health risks and securing informative data for enabling efficient and effective clinical decision making. In real-time, multiple datasets from one or more subject-mounted devices can be collected. The different formats of the collected data can be standardized, by (for example) ensuring that each format complies with a standard, such as Health Level Seven (HL7) or Fast Healthcare Interoperability Resources (FHIR). The data can then be transmitted to a centralized cloud platform using a secure and/or real-time data transmission protocol. Furthermore, AI-based health risk prediction models can be used to predict the risk on the transmitted healthcare data of a subject. The system may be augmented by a decision support system for healthcare providers to generate automated alerts in case of emergency events via user-interfaces for both health service providers and subjects.

Embodiments of the disclosure can reduce bottlenecks that hamper information sharing, care coordination, and subject engagement in hospital settings by fusing a centralized cloud platform with customed services that are hosted in it. Currently, care providers focus on the curative care rather than on the preventive care, which can lead to a higher number of preventable hospital visits and increased cost to their subjects for availing hospital services. Some embodiments of the inventions provide information and/or infrastructure to support healthcare practitioners to provide more customized treatments, better predict prospective health issues, and support developing collaborative connections with subjects by combining the electronic health data, preferences and feedback into a single platform. The obtained information enables effective predictive analysis against every subject. An advanced healthcare system is presented that is centered on a subject by integrating its profiles with a smart cloud-based technology. This provides an opportunity to elevate healthcare, improve subject outcomes, improve operational efficiencies, and transform healthcare experience.

To address challenges faced by the health care providers regarding the response of a subject towards the services provided, feedback for the patients can be collected through various sources such as online portals, feedback forms, surveys, and social media. A computer-implemented method can be provided that includes receiving, via various applications and channels, feedback data from subjects, where feedback is responsive to interactions of subjects with services; employing one or more natural language processing (NLP) techniques to preprocess the feedback data, extracting a subset of the feedback where the subset represents textual and contextual information, utilizing one or more machine learning models e.g., sentiment analysis to gauge whether feedback is positive, negative or neutral and topic modeling to automatically categorize the subset of the feedback into areas that indicate a type of the patient experience such as clinical care, communication, accessibility and the like and assigning corresponding scores to each service delivered. Additionally, one or more anomaly detection algorithms can be applied to identify areas to flag within the categorized feedback, with a focus on detecting patterns that are indicative of potential issues or negative experiences. Further, conducting statistical analysis and data visualization to gain deeper insights into the experiences of subjects and the performance of the services are provided. This may involve trend analysis, correlation studies, and anomaly identification.

A computer-implemented method may be integrated that provides security to real-time communication between a device of subject and a device associated with a health care provider by using of a subject-centric smart system that introduces a secure channel of communication with guardrails to comply with security constraints, such as complying with HIPAA rules. These guardrails protect subjects' personal health information in the healthcare ecosystem. Unifying Humanitarian Device Exemption (HDE) gateways between the subject and the provider interfaces enable improved care delivery. It also reduces the cost of the healthcare delivery and the probability of medical errors including but not limited to misdiagnosis and mistreatment. Furthermore, customized care delivery can be used to generate the persona of each subject on the social and medical history that may effectively improve the satisfaction of a subject as it helps in providing customized care delivery for every subject.

A persona of a subject may be generated by combining electronic healthcare data based on some or all of online footprints of a subject including real-time communication channel and medical records (e.g., where data retrieval or filtering is performed selectively to accord with internal or standard privacy protocols). Smart hospitals can introduce systems for tracking and predicting the health of a subject using subject-mounted devices and electronic healthcare data recorded in a subject health application by using machine learning models. A health record of a subject can be used to monitor data of the subjects in real-time, which can help identify and track health patterns to inform clinical decision making.

Smart hospitals can use one or more models for predictive and prescriptive analysis based on the data driven decisions across clinical, administrative, and business processes. Analytical models can be integrated to the software of the system for decision making in smart hospitals. This not only improves the workflow efficiency but also reduces the cost incurred and improvement in patient satisfaction. The system may integrate voice scribe services in the interfaces of subjects and health providers to avoid unnecessary delays in recording the healthcare data of subjects. The voice scribe service may provide intelligent interpretation of conversation and automatically recording the healthcare data of subjects in an electronic healthcare record. It may also support various document formats such as standard (for clinicians), subject-consumable format or a referral report.

Various disparate systems that manage separate components result in ineffectiveness in management and customization of services provided by a hospital. The smart hospital, as disclosed herein, employs a smart building using internet of thing (IoT) sensors and devices that provide a centralized system within the hospital reducing the cost of implementation. The system may integrate an augmented/virtual reality (AR/VR) interactive service for training and education of doctors to avoid complex, costly and time-consuming training procedures. The seamless integration of AR/VR services provide benefits from clinical care to patient education and staff training for pre-procedural counseling of subjects or medical personnels to spread awareness and avoid poor experience of subjects. The present disclosure may integrate a computer-implemented method, facilitating remote video consultation with subjects of an infectious disease, automating routine tasks using robots for supplying food or medication to subjects in a healthcare facility and avoiding high risk of mortality in subjects because of exposure to infectious organisms in hospital wards. This reduces infection rates among the medical staff, improves efficiency and optimizes human resource utilization. The automation of tasks may also increase utilization of nurses and auxiliary staff to avoid performing mundane and repetitive tasks. In some embodiments, a computer-implemented method is provided that includes: collecting data in real-time from one or more data sources for one or more subjects; aggregating the data of each of the one or more subjects with associated one or more electronic medical records to build a persona for each of the one or more subjects; deploying at least one service within each sublayer of a subject-centric matrix, wherein each sublayer of a subject centric matrix is customized to include the at least one service for a particular healthcare facility, wherein a service is customized according to the persona associated with each of the one or more subjects, and wherein the service includes a data processing module configured to collect and store data, a communication interface module configured to exchange data, and an artificial intelligence-based engine to perform data analytics on collected data, and wherein the subject-centric matrix includes: a first sublayer including the one or more first services to know the subject by collecting the data from one or more electronic medical records for creating the persona associated with the subject, or providing a real-time communication between a plurality of subjects; a second sublayer including the one or more second services for collecting the data from one or more subject-based applications or one or more subject-mounted devices for predictive and prescriptive analysis of the one or more subjects within and outside the healthcare facility; and a third sublayer including the one or more third services that are based on data-driven and artificial intelligence for enhancing experience of the one or more subjects, the one or more third services include quick registration, augmented/virtual reality (AR/VR), voice scribe, building with internet of things (IoT), or real-time tracking of the one or more subjects; and deploying a cloud platform configured to host the services, wherein the cloud platform includes: an interoperability module configured to synchronize a plurality of disparate datasets for establishing data exchange between a plurality of healthcare facilities; an integration module configured to provide a consistent data format and exchange of the data between sublayers in the subject-centric matrix; and a customization module configured to customize the one or more services for the particular healthcare facility and for a particular subject according to the persona associated with the subject.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an exemplary overview of a smart hospital system and a cloud services platform provided to multiple subjects in a centralized subject centric matrix.

FIG. 2 shows an illustrative example overview of an architecture of a smart hospital that consists of sublayers embedded on a cloud platform.

FIG. 3 shows an example of a layered architecture of a subject centric smart hospital integrating smart services on a cloud platform and providing interoperability for integrated systems.

FIG. 4 shows an exemplary modular structure of the services interacting with the cloud databases solving data disparities.

FIG. 5 shows an exemplary flow of data between different services integrated into a centralized cloud platform in a subject centric smart hospital.

FIG. 6 shows an illustrative breakdown of a cloud platform including one or more servers, gateways, databases, and object storage services.

FIG. 7 shows an illustrative example of an interaction among users in a local area network and a wide area network over a cloud that provides multiple services to the end users.

FIG. 8 shows an example end user device interaction with a hospital server.

FIG. 9 shows an example diagram explaining the interoperability between different smart hospitals connected through a cloud via internet.

FIG. 10 shows a flowchart illustrating an example flow of customized services provided by a cloud to a smart hospital in accordance with a client and subject preferences.

FIG. 11 shows an example flow of procedures for a subject in a subject centric smart hospital.

### DETAILED DESCRIPTION OF THE DRAWINGS

The system includes a system architecture that leverages cognitive computing technologies in real-time where the cognitive computing technologies uses artificial intelligence and other techniques to provide increased automation, boost user engagement, promote integration and interoperability across siloed hospital information systems. Certain aspects of the present disclosure include a subject-centric matrix in a smart hospital system where multiple sublayers of smart services can interconnect with each other in a healthcare ecosystem to provide an embodiment of a data-driven, subject-centric medical stack. A healthcare facility can choose one or more services in a service layer without the need for a prerequisite service to be in the place first. The smart services in this matrix can be hosted on a cloud platform that may offer an integration module for transforming data from disparate sources into a standardized format for data consistency and compatibility. The transformed data is stored in a centralized cloud platform such as Microsoft Azure, Amazon Web Services (AWS), or Oracle Cloud Infrastructure (OCI), making it accessible for analysis and processing. The integration module manages application programming interfaces (APIs) to enable communication with different smart services. APIs allow these services to request and exchange data with the integrated data repository. The cloud platform may have a customization module that can enable hospital administrators or healthcare providers to add or remove services based on the preferences of a healthcare facility or a subject. This may include a user-friendly interface for managing services. When a new smart service is added into a sublayer of a subject-centric matrix, the cloud platform with the help of an integration module may configure data connections and APIs to seamlessly integrate it into a cloud platform. The integration module governs the flow of data between integrated services and the central data repository providing that the data is secure, meets privacy regulations and is routed appropriately to the endpoint components in a healthcare stack. Hospital or subject preferences may trigger the addition or removal of healthcare services. The integration module dynamically updates the platform to reflect these changes without disrupting ongoing services. The module is designed to scale with the addition of new services or increased data volumes, ensuring the performance and responsiveness of the healthcare platform.

In an embodiment of the present disclosure, a data interoperability module can be provided in a cloud platform for managing various workflows and data heterogeneity from different health centers operating in silos that results in data isolation, loss of operational efficiency affecting the quality of a subject care. The interoperability module may include Extract, Transform, Load (ETL) processes for extracting data from different sources such as health centers, transforming it into a standardized format, and loading it into a centralized storage medium or data repository. The standardization of data may be done by mapping and converting data into a common scheme or format, adhering to industry standards like Health Level Seven (HL7) or Fast Healthcare Interoperability Resources (FHIR). The system establishes data transmission protocols by implementing secure data transfer methods such as HL7 messaging, RESTful APIs, or other healthcare-specific protocols to facilitate batch or real-time data exchange. The data governance practices can be implemented to maintain data quality, security, and compliance with the regulatory requirements such as HIPAA by creating policies, roles, and responsibilities for data management. Further, subject identification and record matching can be performed by a Master Patient Index (MPI) that involves creating a unique identifier for each subject, steady across all healthcare centers and healthcare exchanges providing accurate aggregation and integration of records of a subject. Subject data can be protected by doing data encryption, using access controls, and doing audit trails to secure data in transit during transmission and at rest in the storage for complying with the privacy regulations and to get the trust of a subject. The integration with a Health Information Exchange (HIE) system can be done to facilitate secure data sharing between healthcare centers serving as intermediaries that manage consent and enable data sharing.

The system may also leverage analytics and reporting tools to extract meaningful insights from the processed data for making informed decisions to improve the quality of care provided to a subject. The system capabilities can be enhanced by monitoring interoperable systems with regular audits, performance assessments, and feedback mechanisms to identify issues, provide data quality, and make continuous improvements. Additionally, the system can be made scalable to accommodate future data sources that are upgraded with the emerging interoperability healthcare standards.

The present disclosure may provide services integrated in a cloud platform related to various medical procedures for a hospital through a user terminal (for example, a smartphone of a hospital user or a dedicated terminal provided in a hospital), thereby reducing travel, and waiting time at a healthcare facility for undergoing a medical procedure. As an illustrative example of the present disclosure, subjects entering a smart hospital are greeted by a digital kiosk that maybe equipped with the facial recognition technology or a subject is asked for a QR code or biometric verification. Through this automated identification, the medical records of a subject are retrieved in nearly real-time, and the registration process is completed quickly. This eliminates the need to provide data on a paper and reduces the wait time of a subject. After registration, the subjects are directed to the respective waiting areas where they receive real-time updates on their waiting status in a queue via a mobile application. This application can also provide estimated wait times and communications of a subject with the hospital staff if a need arises. Once waiting, the subjects may use augmented reality (AR), or virtual reality (VR) headsets provided by a smart hospital. These headsets offer a variety of treatment procedures, healthcare information, and entertainment options from immersive games to tranquil virtual environments, help in reducing the anxiety, empower subjects and improve the overall waiting experience once waiting in a queue of a hospital. If, for example, a subject is wearing a subject-mounted device such as a smart wearable device that monitors subject vitals (e.g., heart rate and blood pressure). This data can be transmitted to the central monitoring system of a smart hospital in real-time. If any abnormalities are detected, healthcare providers are alerted immediately, allowing for a quick intervention. During the stay of a subject, the subject may encounter robotic assistants in various roles. For example, a robotic nurse delivers medications and supplies to the rooms for the timely delivery of supplies to the subjects, thereby reducing the workload of human nurses. Robotic cleaning staff members also maintain hygiene standards in the common areas. The hospital may also utilize sentiment analysis software to collect feedback of subjects in real-time and assess their emotional state during their visit. Subjects are periodically prompted to share their thoughts and feelings during their stay in the hospital. If any negative sentiments are detected, hospital staff may intervene to address the relevant concerns and improve the subsequent experience of a subject.

To enhance the experience of a subject further, the subjects may receive automated appointment reminders and personalized healthcare tips through different applications of a smart hospital. To incentivize healthy behaviors, the application can offer rewards and discounts for adhering to the prescribed treatment plans, showing up for the follow-up appointments, and participating in different wellness programs offered. Smart sensors and IoT devices in the rooms of subjects can automatically notify housekeeping staff when cleaning is needed. Additionally, subjects can use the hospital application to request a room service, adjust the temperature of a room and lighting, and control their entertainment options, thereby enhancing their comfort and hence satisfaction during their stay at the hospital. By integrating these smart services into the operations of a hospital, the subjects may experience reduced waiting times, high quality yet personalized healthcare services and management, persona specific education and entertainment content, and improved communication, which can enable value based, data driven personalized healthcare delivery platform and services. This may lead to an enhanced satisfaction level of a subject, better health outcomes, and overall, an efficient, pleasant, and satisfying healthcare experience at a smart hospital. Furthermore, a smart hospital can choose the integration of all, additional or any one of these services mentioned in the above within the layered architecture of its medical stack.

The subject-based applications for predictive analysis in a smart hospital can be numerous, as they aim to enhance the care of a subject, streamline hospital operations, and improve overall efficiency. An example may include subject health monitoring applications that monitor subject vitals (e.g., heart rate, blood pressure, temperature) and use predictive analytics to identify potential health hazards. The alerts may be sent to healthcare providers in real-time. Another example may include readmission risk prediction i.e., the risk of readmission of a subject after a discharge from the hospital. By analyzing the historical data and the characteristics of subjects, hospitals allocate resources effectively for a follow-up care. Smart applications, with the help of camera feeds, can analyze the mobility data of a subject to predict its risk of falling. Subjects at a higher risk may receive special attention to enable compliance with precautions, recommended by a physician, to prevent subsequent accidents. Similarly, medication adherence applications can predict whether a subject is following the medication schedules prescribed by a physician. To enable this, the system may send reminders to subjects and notify healthcare providers in case of non-compliance. Staff scheduling applications predict subject admission rates so that staff can be effectively planned and allocated to different services. This enables the staff adequacy in hospitals during peak periods that may reduce costs once the expected occupancy of beds is below the average levels. Furthermore, the applications that perform predictive analytics are used to identify potential outbreaks of infections within a hospital. Monitoring data related to infections and symptoms of subjects can help the management of hospitals to take proactive measures to prevent the spread of diseases.

As described above, the present disclosure provides a customizable subject-centric matrix that enables the customization of smart services according to the persona of a subject. As an illustrative example, subject X enters a smart hospital. On arrival, the subject checks in at a self-service kiosk. The kiosk can recognize the unique identification of a subject, which is linked to the persona profile of a subject in the smart cloud platform of the hospital. The persona profile of subject X is based on the medical history, preferences, and previous encounters with different service providers of the hospital. The smart cloud platform may categorize him as a “Tech-Savvy Senior” due to the age and preferences of the subject for digital health management tools. As a “Tech-Savvy Senior,” the experience of subject X is tailored to the subject persona such that the kiosk interface displays larger fonts and simplified navigation to cater for enhanced visual aid related to the age of a subject. The subject receives notifications through the mobile application of the subject, which the subject prefers over an email or an SMS. The smart services accessible to him include video consultations with doctors, digital prescription management, and daily health tips via an application. The application interface can offer voice commands to enable a conversational experience with different applications in the medical stack of a smart hospital. During a subject's appointment, the doctor may use IoT-connected medical devices to monitor vitals of a subject X. These devices transmit real-time data to the smart cloud platform, which analyzes the health profile of a subject and update its persona. The smart cloud platform, considering the persona of Subject X, generates personalized health recommendations. It suggests relevant articles and resources on topics like wellness, home exercise tutorial videos, and nutrition. The doctor of subject X prescribes medication, and the prescription is digitally sent to the mobile application on the smartphone of the subject. The application can provide medication reminders based on the persona preferences for notifications and reminders. Subject X provides feedback pertaining to his experience in the hospital by using this application. The subject may indicate that a quieter waiting area is preferred, and this feedback is added to a persona profile of the subject. The persona profile of a subject X is updated based on the feedback collected in every interaction, encounter, and experience. The hospital uses this information to refine and customize plans for a future experience of the subject enabling an alignment in healthcare journey of a subject with the subject persona.

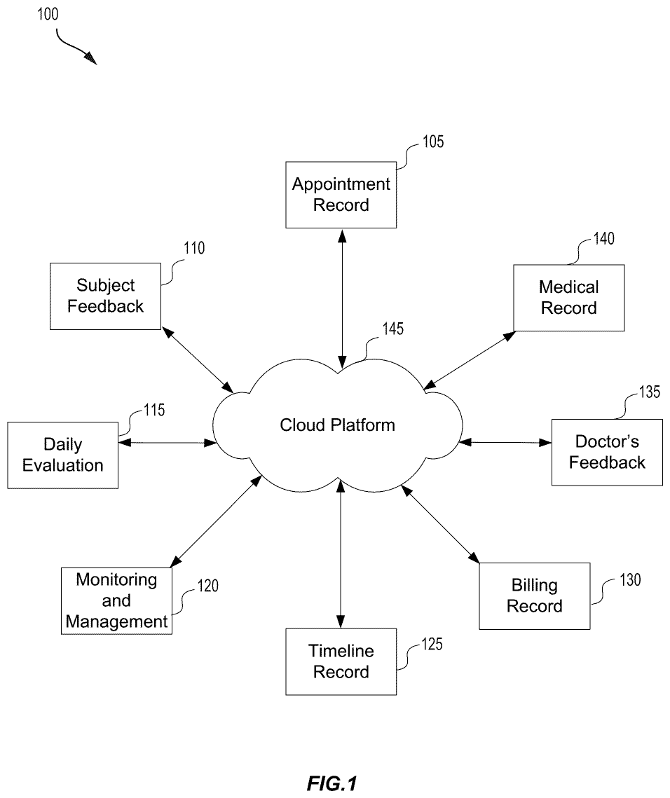

FIG. 1 shows an exemplary overview of a cloud platform 100 providing various services in a subject centric smart hospital. The system comprises of services that can be hosted independently on a cloud platform 145 collectively performing work under one platform. The cloud platform is a cutting-edge centralized cloud-based solution that can act as a brain of contemporary healthcare institutions. The cloud platform 100 enhances a subject experience in a smart hospital by combining various services in an intelligent healthcare environment. It is configured to provide a variety of services that are dynamically customized according to the preferences of a subject, a healthcare facility. or a medical personnel. The cloud platform may offer services and data integration such as appointment record 105, medical record 140, doctor's feedback 135, billing record 130, timeline record 125, monitoring and management record 120, daily evaluation record 115, and subject feedback record 110 for a subject centric smart hospital system. The services aim to assist subjects during the pre-hospital, in-hospital, and post-hospital experiences. The appointment record 105 can include the record of a scheduled meeting or an on-line consulting session of subjects with relevant service providers. It may include the name of an individual, appointment date, or the purpose, which helps to manage and track appointments efficiently. Medical records 140 may contain the clinical record and reports of a subject, including the medical history, diagnoses, pre and postoperative care, treatments, and test results that may enable healthcare professionals to deliver evidence based personalized quality care. Medical records 140 facilitate communication between various medical professionals for the continuity of healthcare.

Doctor's feedback 135 can offer information about the condition of a subject and whether the subject is taking the prescribed medications, suggestions for a therapy, and precautions after the subject is discharged from the hospital. This input creates an effective and efficient follow up plan to provide care to each subject including adherence to prescribed treatment programs. The billing records 130 may determine the charges for services provided to a subject and it systematically stores the payments made by the subject during each visit by keeping a log of the billing details and provide insights into the outstanding balance of a subject. The medical history of a subject can be outlined in a chronological order in a timeline record 125, which can include incidents, diagnoses, treatments, and interventions including medical procedures. For healthcare providers, it offers a concise summary of the health journey of a subject. Additionally, the monitoring and management records 120 include the management and analysis of the services delivered to a subject and the data related to subject health monitoring in various departments of a healthcare facility. It aids in monitoring ongoing surveillance of the health state, vitals, and treatment progress of different subjects. It further keeps a log of the resources and assets provided to a subject, for example the bed and other medical equipment, thus aiding the hospital's asset management system. Furthermore, daily evaluation 115 can be obtained from real-time tracking of the subjects to determine the health status of a subject and correspondingly updating the health record of a subject. The daily evaluations further help the medical personnels to determine the accuracy of a diagnosis and the prescribed medicines. Lastly, subject feedback 110 can be obtained by conducting interviews with the subjects, filling out feedback forms or using biometrics which provides the feedback of a subject about the quality of services and treatments during the stay of a subject in a hospital which further aids in improving the hospital services and the subject-centered care.

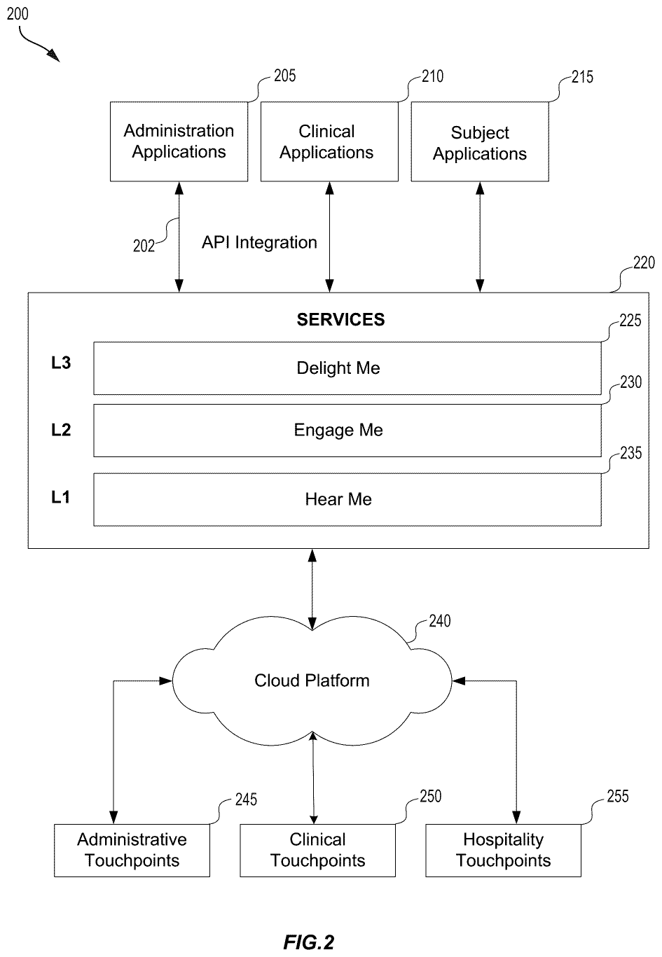

FIG. 2 shows an illustrative example overview of a smart hospital 200 as proposed in the disclosure. The smart hospital includes an application layer, a services layer 220 that is hosted on a centralized cloud platform 240, and a care delivery system. The application layer further includes but not limited to administration applications 205, clinical applications 210, or subject applications 215. Administration applications 205 can manage operations of a hospital and do registration of subjects; clinical applications 210 can maintain the medical records of the subjects and assist in scheduling appointments with medical personnel, and the subject applications 215 may enable navigation across a healthcare facility and provide delivery services to the subjects such as medicine and food. The application layer is linked with the services layer through API integration 202. The services 220 are distributed in a subject-centric matrix further comprising of sublayers, each providing different services to the subjects and the providers. The three-layered subject centric matrix may provide dynamic customization of services for various health care facilities ranging from small sized hospitals to multi-specialty hospitals, wherein the services are added or removed depending on the preferences of a health care facility. The first sublayer, L1, is the hear me layer 235 including smart services that are configured to know and understand the subjects. The second sublayer, L2, is engage me layer 230 including smart services for engaging the subjects both within and outside a hospital. The third sublayer, L3, is the delight me layer 225 including smart services designed to delight the subjects by providing a quality and personalized hospital experience. Cloud platform 240 provides various modules to facilitate the integration and customization of the services. It may provide interoperability between siloed departments within a healthcare facility and between various healthcare organizations. The cloud platform 240 can be further used for deploying one or more services in each sublayer for a care delivery system that comprises of administrative touchpoints 245, clinical touchpoints 250, and hospitality touchpoints 255.

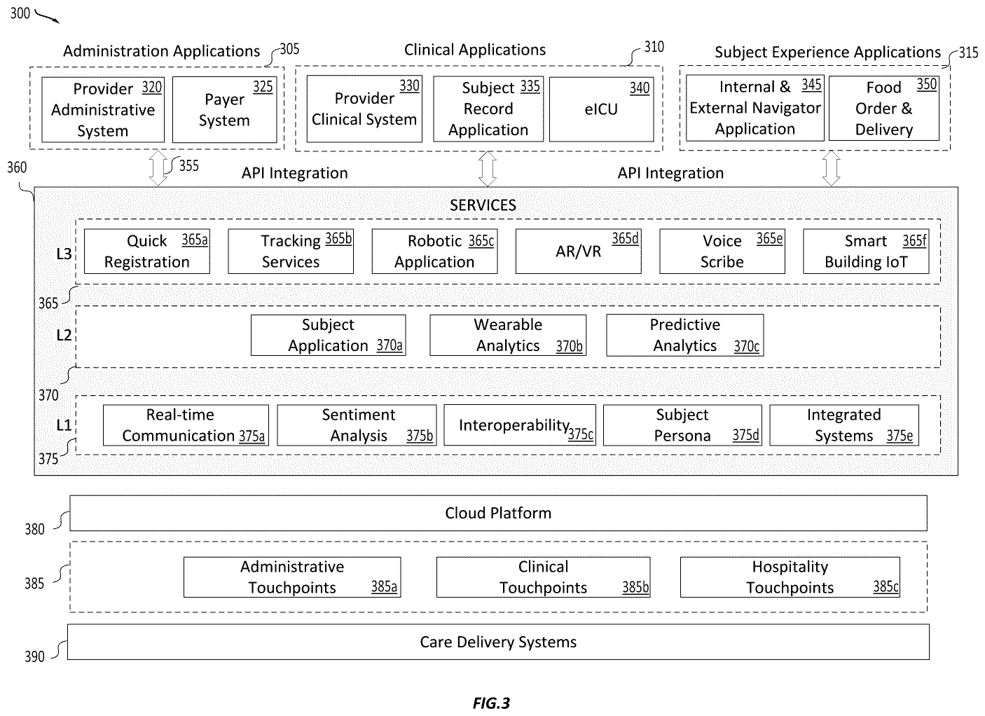

FIG. 3 shows an example layered architecture of a smart hospital 300 comprising of an application layer, a services layer 360 that are hosted on a cloud platform 380 and a care delivery system 390. The application layer further comprises of various applications such as administration applications 305, clinical applications 310, and subject applications 315. Administration applications 305 may include a provider administrative system application 320 that is used by the administration of a health care center for registering the subjects and a payer system application 325 for managing the bills and payments of subjects. The clinical applications 310 include various applications such as a provider clinical system application 330, a subject medical record application 335 and an eICU 340 application, used by clinics to register a subject and schedule appointments with the medical personnel, maintain health and medical records of subjects, and provide support and care including remotely at the place of residence. The subject applications 315 may include an internal and external navigator application 345 and a food order and delivery application 350 that aids a subject to navigate across a healthcare facility and provides hospitality services to the subject. The applications are interconnected with the services through APIs 355 enabling communication between two or more applications. The APIs 355 act as a software intermediary that processes the data transfer between the application layer and the services layer 360. Some of the examples of APIs are RESTful APIs, WebSocket APIs, RPC APIs or SOAP APIs.

The services 360, hosted on a cloud platform 380, are structured in a subject centric matrix comprising of sublayers: L1 375, L2 370 and L3 365 where L1 layer 375 is designed for knowing and understanding subjects better. The L1 layer 375 may include services such as real-time communication 375a, sentiment analysis 375b, interoperability 375c, subject persona 375d, or integrated systems 375e. The real-time communication 375a services can obtain updates on preferences of a subject and gain insights by analyzing the updated information related to a hospital, enabling HIPAA compliant communication between the subject and the medical personnel. The sentiment analysis 375b may involve monitoring and analyzing the sentiments of subjects regarding the services provided and the procedures conducted by a healthcare facility. This is triggered by analyzing feedback forms of subjects to get an objective analysis of the feedback of subjects regarding the services; consequently, a score is assigned to each service. Interoperability 375c may serve integrity of the data of subjects by providing seamless flow of the medical information of a subject across multiple health care centers. Subject persona 375d can be created from the medical history of subjects, the demographic details, socio-economic status, and insurance information that is obtained from billing footprints, real-time communication, and medical records. The obtained subject persona is used for customized care delivery. The integrated systems 375e may include various siloed healthcare facilities having data of a subject. Integration and communication between various applications such as the personal health record (PHR) applications and electronic medical record (EMR) applications can enable data flow between different systems.

The sublayer, L2 370 may involve engaging subjects both within and outside the premises of a hospital. This can include data collection through the subject applications 370a and wearables-based analytics 370b. Subject-mounted devices or wearables such as smart watches, patches and insulin pumps obtain quality of health indicators of a subject, and the state of the subject health may be inferred from them by running analytical tools. Another aspect of engaging subjects may include prescriptive and predictive analytics 370c that provides proactive/predictive alerts, parameter trends, useful health tips and targeted health education. In some instances, the wearables-based analytics 370b attains a preventive approach rather than a curative approach that can reduce the number of encounters and the hospital visits.

The final sublayer L3 365 focuses on the personalized experience of a subject by providing services such as quick registration 365a through a number of smart identification technologies (e.g., biometric, facial recognition, and for remote users a QR code) and storing them into a database. Furthermore, real-time tracking services 365b track geolocation of a subject inside a healthcare facility and assist the subjects to reach their desired location. The services may also be used to track medical personnel and the assets of a hospital for efficient utilization of resources. Robotic applications 365c automate the routine tasks resulting in reduced infection rates among the medical staff. Robotic applications 365c can enable remote video consultations, food or medications delivery to the subjects in a hospital and assisting senior citizens in their desire for a safe mobility. Furthermore, interactive virtual reality-based technologies such as AR/VR technology 365d can be used for training and educating the subjects for a better knowledge of medical procedures. The voice scribe technology 365e that automatically extracts relevant medical information from subject-provider conversations and stores them in medical forms and/or medical records; consequently, a provider can spend more time interacting with the subjects to bring an element of empathy and humanism in providing value based personalized care. Lastly, smart building IoT 365f may provide customized and centralized management. Various IoT devices can be employed for automating repetitive routines. The services can be customized according to the requirements of different healthcare facilities. The services on the cloud platform 380 can be provided through various care delivery systems 390 comprising of but not limited to administrative touchpoints 385a, clinical touchpoints 385b and hospitality touchpoints 385c. The administrative touchpoints 385a may include registration, billing, insurance claims processing and smart management of the queue of subjects at a registration kiosk to significantly reduce the wait time. The clinical touchpoints 385b may include medical records and the demographics data of subjects. Lastly, the hospitality touchpoints 385c may include real-time location tracking services in a smart hospital.

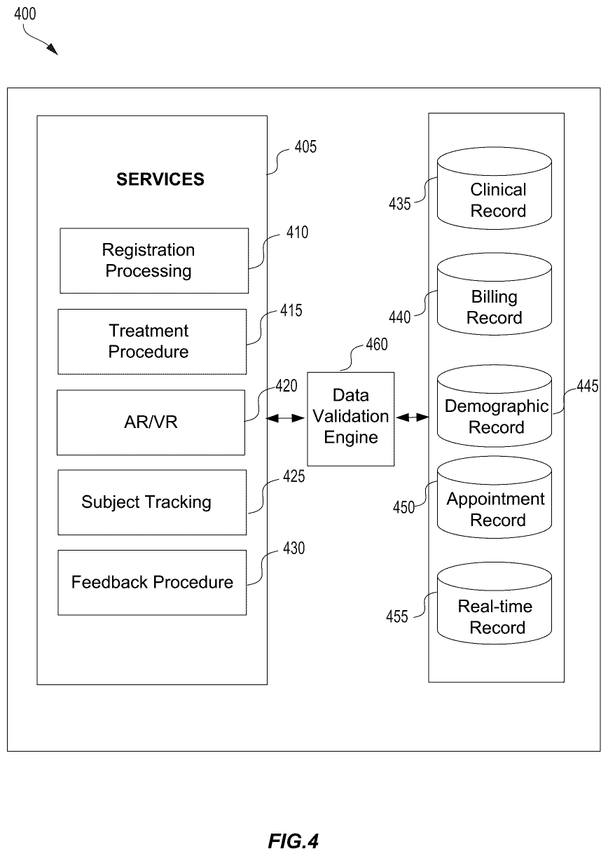

FIG. 4 shows an exemplary modular structure of a cloud platform that hosts one or more services 405 deploying a data validation engine 460 to store the data of subjects into various databases. The data validation engine 460 can be used to verify that the data received from the services is compliant with the format of the data stored in the databases. The data validation engine 460 may detect data duplication and inconsistencies in the medical data before storing it into the databases. It may provide the integrity of data transmitted between the services 405 and the databases in the cloud platform. The databases are used to store records such as the clinical record 435, billing record 440, demographic record 445, appointment record 450 and the real-time record 455 of a subject. The cloud platform can host various services such as the registration processing 410 that retrieves the data of multiple subjects from one or more databases for registration. This may include accessing the personal information of a subject from a demographic record 445 database, while the medical records of the subjects can be accessed from a clinical record 435 database. The treatment procedure 415 may use the clinical records 435 containing the medical history of the subject. It further obtains data from the appointment records 450 database so that the date of a procedure is consistent with the appointment date of the subject with the relevant medical personnel. The AR/VR 420 may use the clinical records 435 or the information of a subject that can be stored in the demographic records 445 database to facilitate a medical or surgical procedure by educating the subject about the planned procedure in a virtual and/or augmented reality environment. The subject tracking 425 may use in real-time the healthcare data of a subject that is stored in the real-time record 455 database to assist the medical personnel in correctly recognizing a subject or other medical staff. It also helps the subject to find the location of any department within a healthcare facility. Lastly, the feedback procedure 430 may record the feedback of a subject related to the various services using real-time communications data that can be stored in the subject real-time record database 455. The data validation engine 460 validates the data shared between the services and the databases on a cloud.

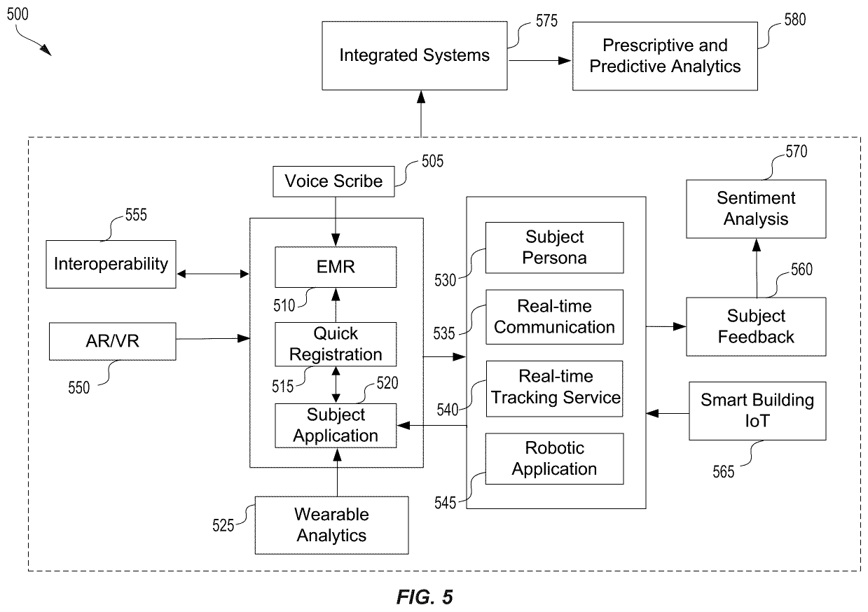

FIG. 5 shows an exemplary flow of data in a subject centric smart hospital 500 between various services integrated in a cloud platform. First, the data of a subject can be collected from one or more databases to build a subject specific persona to provide services in accordance with the preferences of the subjects. In some instances, the services such as voice scribe technology 505 facilitates intelligent interpretation of conversation where the voice scribe 505 may use a subject or provider interface applications or robots to automatically extract the relevant data of a subject from the conversations between a subject and its providers and store it into its medical or healthcare records. This can enable the providers to spend more time with the subjects and show empathy about their health status, as they do not need to spend time manually entering the large data of a subject into a complex medical record system. The data extracted from the voice scribe technology 505 facilitates in maintaining an Electronic Medical Record (EMR) 510 of a subject, wherein the EMR 510 is a digitized version of the contact details of a subject, associated medical history, test results, diagnosis, procedures, and the corresponding treatment that is prescribed by a physician to a subject. The EMR 510 can further provide treatment, progress and diagnoses plans such that the details are updated after each encounter in a smart hospital with the medical personnel.

In some instances, wearables-based analytics 525 collect data through portable devices such as watches and insulin pumps and analyze the data through analytical tools to gain insights on the health status of a subject. The wearable analytics 525 can further assist in predicting health risks based on the collected data, even enabling remote monitoring of subjects. This helps in significantly reducing the frequent visits to hospitals resulting in a preventive approach, by using subject applications 520, rather than the curative approach. Subject applications 520 allow the subjects to access their health information, track their appointments, set reminders for medications, and receive feedback from the healthcare providers. The applications are also used for diet management and sleep monitoring of the subjects. This enhances the engagement of subjects in their healthcare journey by sharing realistically accurate information with the providers in real-time. Hence, subject application 520 provide personalized healthcare service to a subject by reviewing the medical history of the subject.

In some instances, the subject data is obtained through an interoperability module 555 utilizing various subject applications 520 to register the subject into an electronic medical record (EMR) 510. The interoperability module 555 manages various workflows and data disparities from different health centers that are operating in silos that results in data isolation and loss of operational efficiency. The interoperability module 555 also provides uniformity of the data of a subject including the associated medical history, test results and treatment plans and this integrated and fused data reduces the chances of subsequent misdiagnoses. For example, a subject may go for a medical examination to two different hospitals, hence its relevant healthcare data is exchanged through interoperability modules between the two hospitals.

The data obtained through interoperability module 555 helps in quick registration 515 that may be deployed in ambulatory services, in subject services, or referrals within a system. Quick registration 515 facilitates registration and intelligent scheduling of the appointments of subjects by automatically filling details of the subjects. The quick registration 515 may also remotely register a subject, hence reducing the waiting time of subjects. For example, smart hospitals are equipped with digital kiosks with facial recognition technology. The kiosk identifies the subjects, retrieves the medical records associated with the subjects, and completes the registration process relatively within a short time. This eliminates the need for filling a large bundle of papers, hence reduces the registration time of a subject and the wait time of all subjects. Furthermore, medical devices such as vital monitors and infusion pumps are connected to the EMR of the smart hospital. These devices can automatically transmit the health data of a subject, laboratory test results and diagnostic information directly into the medical record of a subject in real-time so that with each encounter of the subject with the healthcare facility, the health record and relevant persona of the subject is updated. In addition to this, AI-driven Natural Language Processing (NLP) algorithms can be used to convert unstructured data, such as physician notes, audio recordings, or handwritten prescriptions, into a structured data that stored as a health record of a subject in an EMR. The NLP technology focuses on interaction between computers and humans through natural language, it uses various technologies to interpret and analyze the written text and/or to transcribe speech into the text.

In other instances, data can be obtained through AR/VR technology 550 where the data is used by subject applications 520 for registration. The AR/VR technology 550 educates and counsels subjects about their respective medical and surgical procedures, where the subject can view and experience in advance the planned medical procedure in a virtual reality environment. Furthermore, AR/VR technology 550 is employed during the training of the medical staff where they can practice in various surgeries in a virtual space using haptic devices that provide a personalized experience. The software for enabling the AR/VR technology 550 is provided in the cloud platform of a smart hospital.

In an example, where the subjects may undergo a complex medical procedure and assuming that the subjects have no prior knowledge of the procedure, so AR/VR technology 550 can provide an immersive and interactive experience to the subjects about the procedure. The subjects wear a VR headset comprising of googles with a screen, and this allows the subjects to not only view the medical procedure in a three-dimensional virtual world but also feel the experience of undergoing through it. In another example, while waiting for an appointment, the subjects may also wear AR/VR technology 550 headsets to have a better understanding of the services offered by a smart hospital. The headsets show a variety of treatment procedures, healthcare information, and entertainment options from immersive games to tranquil virtual environments. This not only aids in educating the subjects but also reduces the anxiety of a subject prior to a medical procedure.

The data obtained from subject applications 520, quick registration 515 and EMR 510 can be used by other services comprising but not limited to subject persona 530, real-time communication 535, real-time tracking services 540 and robotic application 545. The subject persona 530 is created using the information of a subject that includes demographic details, preferences, socio-economic situation, subject data obtained through real-time communication, socioeconomic background of a subject that includes the cultural influences and lifestyle and medical history of a subject. The subject persona 530 can enable the medical personnel to develop strategies for subject-centric experiences where each subject receives customized and tailor-made services that are dynamically updated whenever the subject enters a smart hospital. Moreover, applications on smartphones are used to obtain real-time information of a subject by using real-time communication channel 535 between the subject and the medical staff. Real-time communication channel 535 may update the information about a subject in a healthcare facility by following HIPAA compliant communication between the subjects and the providers.

Furthermore, real-time technologies such as bar codes and radio frequency identification may provide real-time tracking services 540. The real-time tracking services 540 may use mobile applications to track the location of a subject in a healthcare facility and to assist the subject to reach a desired location within the healthcare facility. It can also be used to allocate resources such as beds and medical equipment for the subjects. Additionally, robotic application 545 technology in the cloud platform automates routine tasks such as providing medication and food to the subjects. The data obtained through subject persona 530, real-time communication channel 535, real-time tracking services 540 and robotic applications 545 can be fed into subject applications 520.

Real-time communication 535 obtains feedback 560 of a subject regarding the services and the treatment provided in a healthcare facility. Independent applications are used to obtain the feedback of a subject through the feedback forms, and then benchmarking the services using the quality scores. Furthermore, interviews are conducted with the subjects to obtain their reviews and suggestions. Furthermore, a biometric sensor coupled with the body of a subject is used to tag the feedback. For example, subject-mounted or wearable biometric sensors such as a heart rate monitor is used to measure physiological responses obtained during medical procedures, consultations, or use of healthcare applications. This seamlessly determines the stress level, comfort, or overall satisfaction of a subject. In another example, voice analytics software can be used to assess the tone and pitch of a subject during verbal conversations. This aids in identifying the emotions of happiness, frustration, or stress. Once the feedback is received, a sentiment analysis 570 is performed by monitoring and analyzing the sentiments of the subjects to gauge their satisfaction with the services and the care delivery systems. This helps in inferring the positive and negative sentiments of the subjects and hence take appropriate countermeasures. Thereafter, the data that is obtained from subject persona 530, real-time communication 535, real-time tracking services 540 and robotic applications 545 is utilized in the smart building IoT, wherein various IoT devices are used in ambulatory and in-subject settings. Furthermore, IoT devices control the lighting, temperature, and ventilation system of a room by following the preferences mentioned in the subject persona 530. Moreover, IoT devices provide real-time tracking services 540 to track the movements of subjects within the smart hospital. For example, if a subject has a fever, smart IoT sensors in hospital rooms control the temperature of rooms suited to the health state of the subject.

Finally, the information obtained through the services can be used for prescriptive and predictive analytics 580 that generates personalized healthcare alerts including risk and morbidity predictions and other hazards related to the health of a subject. Various artificial intelligence and machine learning based algorithms can be used to build analytics models for modelling the health state of a subject and predicting in advance the future disease path of a subject. Predictive analytics uses machine learning models to extract knowledge from the EMR, combine and aggregate it with other sources of data to create a unified emerging health person of a subject, and determine the future healthcare needs of the subjects. By using supervised learning models that include but are not limited to linear regression, decision tree, nearest neighbor, random forest, and neural network; or using unsupervised learning models including but not limited to clustering-based algorithms such as K-means clustering, the system can predict adverse outcomes of a subject or the efficacy or effectiveness of a planned procedure or treatment. Prescriptive analytics can help in modelling and solving optimization problems related to inefficiencies and costs of management and administration. For example, issues related to bed occupancy in healthcare facilities may be solved by using optimization algorithms like genetic algorithms to optimize bed allocation and associated costs. Furthermore, the clinical processes can be optimized by using prescriptive and predictive analytics 580 where artificial intelligence based mobile applications can be used to obtain the health information and symptoms of the subjects that aids in creating a unified health map of the health condition of a subject and sending real-time alerts to the medical personnel in a smart hospital.

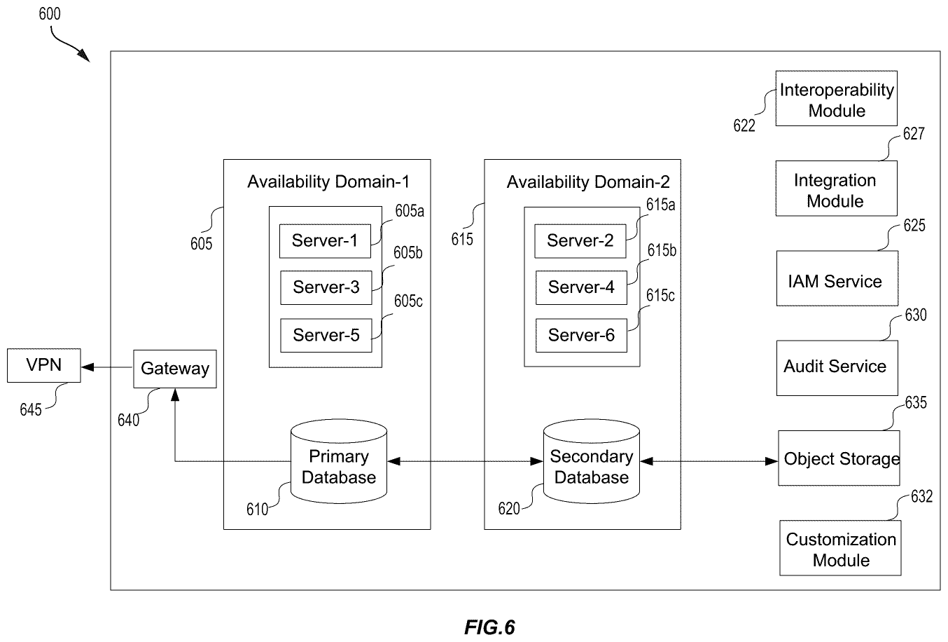

FIG. 6 shows an illustrative breakdown of a cloud infrastructure 600 that acts as a centralized cloud platform configured to host the services for a smart hospital as proposed in the disclosure. The cloud infrastructure 600 comprises of multiple availability domains, availability domain-1 605 and availability domain-2 615 respectively, an identification and access management (IAM) service 625, an audit service 630, an object storage 635, an interoperability module 622, an integration module 627, a customization module 632, a gateway 640 and a virtual private network (VPN) 645.

The availability domain is a physically distinct data center that provides uninterrupted operations between various cloud-based services and applications even in case of hardware malfunction or other failures. Each availability domain is isolated from the other availability domain in terms of the physical infrastructure, power, and networking. The availability domain comprises of multiple servers and databases. The servers in an availability domain are used for hosting applications and services. The incoming network traffic is divided between the multiple servers inside an availability domain. Furthermore, if any one of the servers encounters a failure, the applications are shifted to another server within the same availability domain. The availability domain-1 605 comprises of multiple servers such as server-1 605a, server-3 605b and server-5 605c, it further comprises of a primary database 610. These servers are used for hosting the services. The primary database 610 is a centralized database that stores the data of subjects to facilitate services. It controls the read and write operations and data updates. The primary database 610 allows the services to perform read and write operations where the data is retrieved from the primary database 610 and changes are made to the existing data. The primary database 610 is synchronized with the secondary database 620 that stores a copy of the data in the primary database 610. The availability domain-2 615 comprises of multiple servers such as server-2 615a, server-4 615b and server-6 615c, it further comprises of a secondary database 620. The secondary database 620 is a replicated copy of the primary database 610 and is synchronized with the primary database 610 such that any changes made to the primary database 610 will result in similar changes in the secondary database 620. The secondary database 620 is used for read-only operations where the services can retrieve data from the secondary database 620 but are not allowed to perform the write operations. In case where the primary database 610 encounters a failure the secondary database 620 can act as the primary database 610. Both the primary database 610 and secondary database 620 are secured through authentication, access control and encryption methods.

The object storage service 635 is used to store unstructured data that can be in the form of medical images, videos, medical documents, and subject logs. The data is stored as discrete objects with a unique identifier. The subject data stored in object storage service 635 is accessed by the secondary database 620 by sending a request to the object storage service 635 specifying the unique identifier of an object. Object storage service 635 retrieves the data and sends it to the secondary database 620 where the data is processed and converted to the format compatible with the services. The audit service 630 includes mechanisms and services that can monitor and analyze the activities within a cloud infrastructure 600. The audit service 630 can be used to track events regarding the user interactions and generate logs regarding the user logins, data access and configuration changes. Since audit service enables that privacy and security standards are complied with, they are used to uphold the regulatory compliance requirements such as HIPAA. The IAM service 625 controls the management of user identities and access to cloud services and resources. It is used by healthcare facilities for authorization, maintaining confidentiality and ensuring integrity of the data of subjects. The IAM services 625 allow the healthcare facilities to manage user identities for the authorized users only to have access to the cloud resources. It may comprise of a multifactor authentication to verify the identity of the users before granting access. It is a centralized interface to manage user identities, access policies and permissions across the cloud. It can further control access to the APIs by managing the authentication and authorization for the API endpoints.

The cloud platform offers a data interoperability module 622 for managing various workflows and data disparities from different health centers that are operating in silos resulting in data isolation, loss of operational efficiency affecting the quality of care of a subject. The integration module 627 manages the APIs to enable communication with different services. APIs allow these services to request and exchange data with the integrated data repository. The integration module 627 governs the flow of data that is exchanged between the integrated services and the central data repository. Since the services store and manage data independently, the integration module 627 synchronizes the data across various services, it further provides protocol translation since the services use different yet standard communication protocols and the data formats. The integration module 627 may provide a central interface where administrators monitor and manage data flow, connections, and integration that aids in troubleshooting various problems.

Furthermore, cloud infrastructure 600 includes a customization module 632 that enables hospital administrators or healthcare providers to add or remove services based on the preferences of a healthcare facility or a subject. This often includes a user-friendly interface for managing services. It allows the healthcare facilities to add, remove or modify the existing services. When a new smart service is added into any sublayer of a subject-centric matrix, the cloud platform with the help of integration module 627 configures the data connections and APIs to seamlessly integrate it into the cloud platform.

The gateway 640 can act as an intermediary between the various components of the cloud and the external networks. It allows communication between various systems of healthcare facilities that might use different protocols, hence enabling interoperability. It may also facilitate data routing for optimized data transmission and provide security measures such as firewalls and encryption methods for protecting the cloud infrastructure from unauthorized access and security breaches. The data from the primary database 610 is passed through gateway 640 and then through VPN 645 that establishes encrypted connection between a user device and the cloud resources. The VPN 645 may encrypt the data that flows between the user device and the cloud resources providing network privacy by masking the IP address of users.

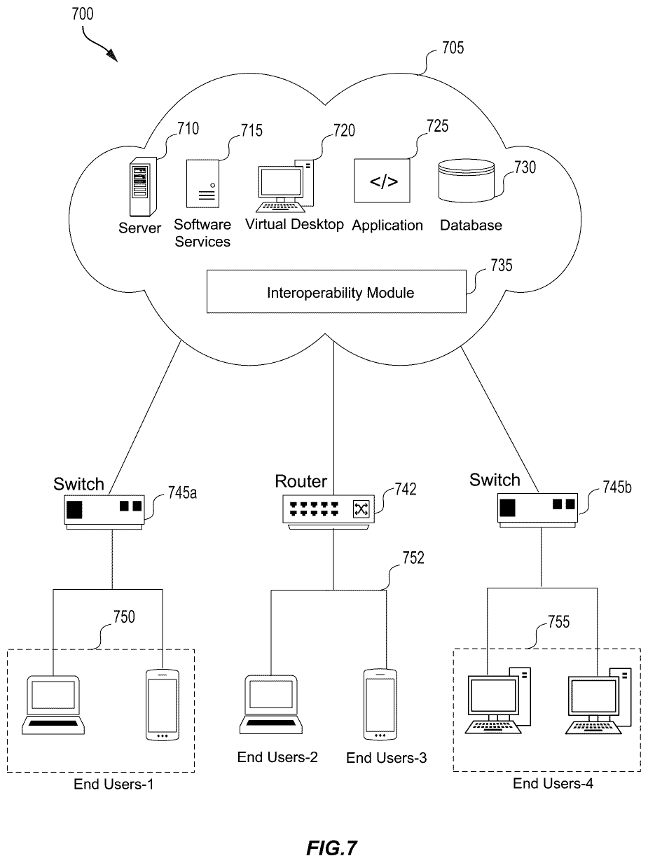

FIG. 7 shows an illustrative example of a system that can enable interoperability between users (inside or outside) of a healthcare facility including end users-1 750 and end users-4 755 (inside the healthcare facilities), end users-2 752 and end users-3 752 (outside the healthcare facility), multiple switches 745a and 745b, a router 742 and a cloud platform 705. The cloud platform 705 may include servers 710, software services 715, virtual desktops 720 applications 725 and the databases 730 for storing the data of subjects. The end user-1 750 and end user-4 755 may represent the logical group of users such as subjects or the healthcare providers that are using a similar LAN inside a healthcare facility, connected through switches; the users such as end user-2 752 and end user-3 752 may represent the subjects or the providers accessing the cloud platform remotely, and hence are connected with the cloud platform through routers 742. After the collection of data from various end users using the applications that may include administration applications 305, clinical applications 310, and subject applications 315, a data validation engine validates the data traffic for the data consistency. The structured data can be sent to the cloud platform 705 that provides a wide range of computing and storage services.

The server 710 (infrastructure as a service—IaaS) can provide virtual desktops 720 and instances of GPU that provide high computational resources in a scalable and flexible manner. Software services 715 (platform as a service—PaaS) provide platform services, development tools and more. These services are used by users to build, deploy, and manage the applications without managing the infrastructure. Various cloud-based applications (software as a service—SaaS) such as customer relationship management (CRM) and enterprise resource planning (ERP) can also be provided. Various database services may also be provided by the cloud platform to increase performance and scalability.

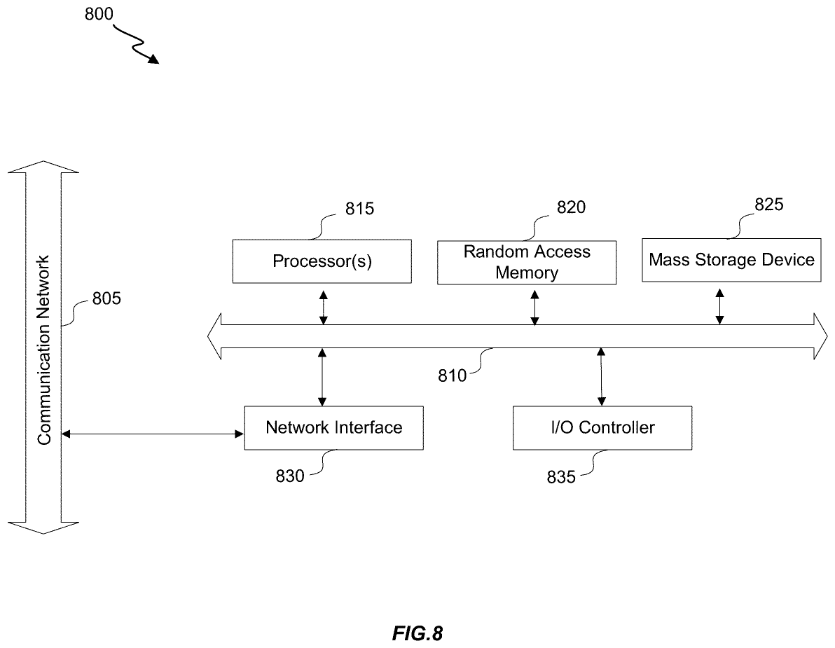

FIG. 8 shows an exemplary end user device 800 (within a smart hospital or outside) that may include a processor 815, a memory (e.g. RAM) 820, a bus 810 which couples the processor 815 and the memory 820, a mass storage device 825 (e.g. a magnetic hard disk or an optical storage disk) coupled to the processor 815 and the memory 820 through an I/O controller 835, and a network interface 830 coupled to the processor 815 and the memory 820, such as a modem, digital subscriber line (“DSL”) card, cable modem, network interface card, wireless network card, or other interface device capable of wired, fiber optic, or wireless data communications. The network interface 830 is further connected to a communication network 805. One example is a personal computer equipped with an operating system such as Microsoft Windows XP, UNIX, or Linux, along with software support for Internet communication protocols. The personal computer may also include a browser program, such as Microsoft Internet Explorer, Netscape Navigator, or Firefox, to provide a user interface for access to the internet. Although the personal computer is a typical end user, it may also be a workstation, mobile computer, Web phone, VOIP device, television set-top box, interactive kiosk, personal digital assistant, wireless electronic mail device, or another device capable of communicating over the Internet. As used herein, the term “end user” is intended to refer to any of the above-described end users or other end user devices, and the term “browser” is intended to refer to any of the above browser programs or other software or firmware providing a user interface for navigating an internet.

In some instances, FIG. 8 illustrates an exemplary server 800 (on either a cloud platform or be an end user at a remote healthcare facility) includes a processor 815, a memory (e.g., RAM) 820, a bus 810 which couples the processor 815 and the memory 820, a mass storage device 825 (e.g., a magnetic or optical disk) coupled to the processor 815 and the memory 820 through an I/O controller 835, and a network interface 830 coupled to the processor and the memory. The network interface 830 is further connected to a communication network 805. Servers may be clustered together to handle more client traffic and may include separate servers for different functions such as a database server, an application server, and a Web presentation server. Such servers may further include one or more mass storage devices 825 such as a disk farm or a redundant array of independent disk (“RAID”) system for additional storage and data integrity. Read-only devices, such as compact disk drives and digital versatile disk drives, may also be connected to the servers. Suitable servers and mass storage devices are manufactured by, for example, Compaq, IBM, and Sun Microsystems. Generally, a server may operate as a source of content and provide any associated back-end processing, while an end user is a consumer of content provided by the server. However, it is appreciated that many of the devices described above may be configured to respond to remote requests, thus operating as a server, and the devices described as servers may operate as end users of remote data sources. In contemporary peer-to-peer networks and environments such as RSS environments, the distinction between end users and servers is a blur. Accordingly, as used herein, the term “server” as used herein is generally intended to refer to any of the above-described servers, or any other device that may be used to provide content such as RSS feeds in a networked environment.

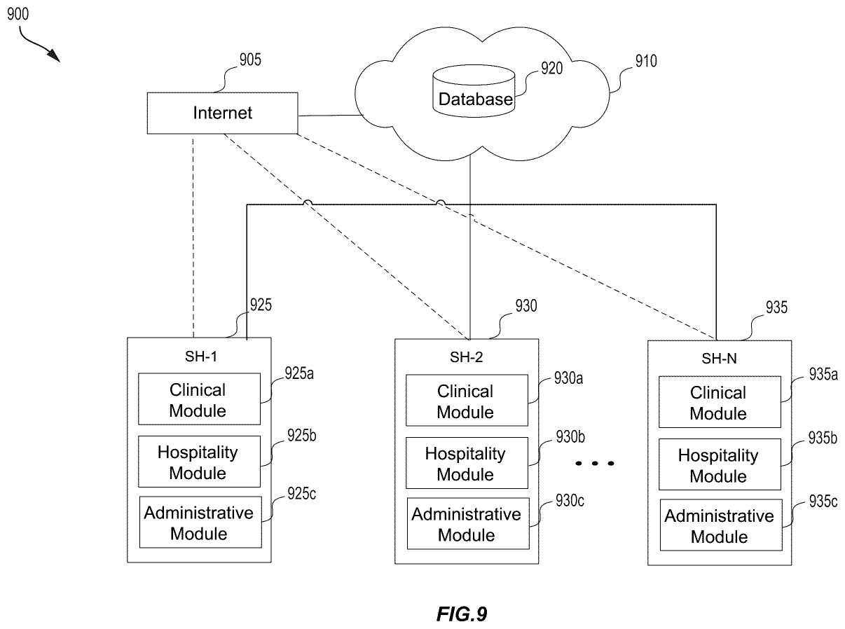

FIG. 9 shows the interoperability framework implemented between multiple smart hospitals for the exchange of data associated with subjects between disparate healthcare facilities. It is a way that can facilitate seamless sharing and exchange of data and services to maintain a coherent and standardized way of communication and collaboration among various health care centers. According to the provided subject centric matrix, the clinical, hospitality and administrative modules can gain access to the subject data that is available in each department through the cloud platform 910 that stores the subject related data in a common database 920. It creates an interconnected system that benefits the health care provider, subjects, clinics, and the healthcare community effectively. The diagram shows the various smart hospitals as a unit such as SH-1 925, SH-2 930 AND SH-N 935. Each unit consists of various modules which are connected to each other i.e., clinical module 925a, hospitality module 925b and administrative module 925c. The units of smart hospitals are also connected to each other through Internet 905 to maintain the data integrity as well as the authentic records of different subjects. The cloud platform 910 provides cutting-edge services to the hospitals as well as the subjects for operational efficiency and better outcomes in a value-based care ecosystem. The various smart hospital units 935n access the data of subjects that are stored on the cloud platform 910 through internet 905. Furthermore, smart hospitals can exchange the data of subjects with one another through cloud platform 910. The cloud platform 910 comprises of software interfaces such as the data validation engine that checks and validates data consistency, accuracy, and conformity. It enables the data consistency and integrity resulting in getting authentic, consistent, organized, and structured data. It defines some validation rules and logic to maintain the validity of data. After the data is structured and organized, then it is transferred to database 920 for storage. A database 920 is a centralized repository for storing the ordered data from multiple matrices and makes the correct data available in real-time to enable effective and authentic communication.

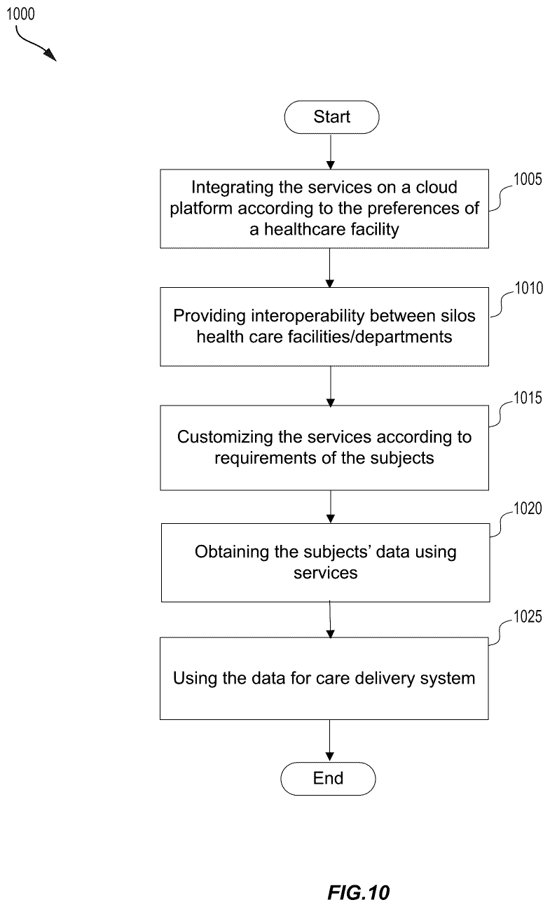

FIG. 10 shows a flow chart illustrating an example flow of a customization of services provided by a cloud platform according to the requirements of different smart hospitals or subjects. The hospitals that have advanced healthcare departments with connected modern equipment may not demand many services to be integrated to the respective systems, so they can customize the subject centric matrix according to their needs. For example, a multi-specialty hospital may already have a subject barcode technology for EMR, so it can customize the services by omitting the quick registration module. Furthermore, the small sized hospitals may choose to analyze the collected data of subjects and identify duplicate data entries so they can deploy services that facilitate the use of an EMR system to enable efficient queue management to reduce the wait times. Moreover, certain healthcare facilities may not have the funds or a suitable architecture to deploy AR/VR technology so they may decide not to use the AR/VR technology in a respective layer.

At block 1005, a subject centric smart hospital integrates the services on a cloud platform according to its preferences. The cloud platform provides interoperability between different departments within the subject centric smart hospital and other remote health care facilities at block 1010. At block 1015, customization of these services is performed in accordance with the preferences and persona of a subject. The cloud offers the customization to personalize the health care experiences according to the preferences, needs and conditions of the subjects. For example, if a subject has an undesirable prior experience with the AR/VR technology and is hesitant to use the AR/VR to have a prior view of a planned medical procedure, so the subject may opt to view it through a healthcare application on a smartphone. At block 1020, the data is collected for each subject through multiple sources such as wearable devices, sensors and devices installed at the health care facility or from the provided services or integrated systems. The collected data is then passed to the care delivery systems that may include centralized EHRs, clinical decision support systems (CDSS), robotic devices, real-time location systems, AR/VR systems, smart infrastructure systems (e.g., HVAC), security and privacy systems for further processing and provision of enhanced subject experience and coordinated care at block 1025. The data can be processed further for predictive analytics to forecast future health events including adverse events, risks and hazards at block 1020. With the help of this proactive strategy, healthcare professionals can manage the quality of care for the subjects by identifying high-risk subjects, tailoring treatments to health status and personas, and adopting preventative procedures wherever possible.

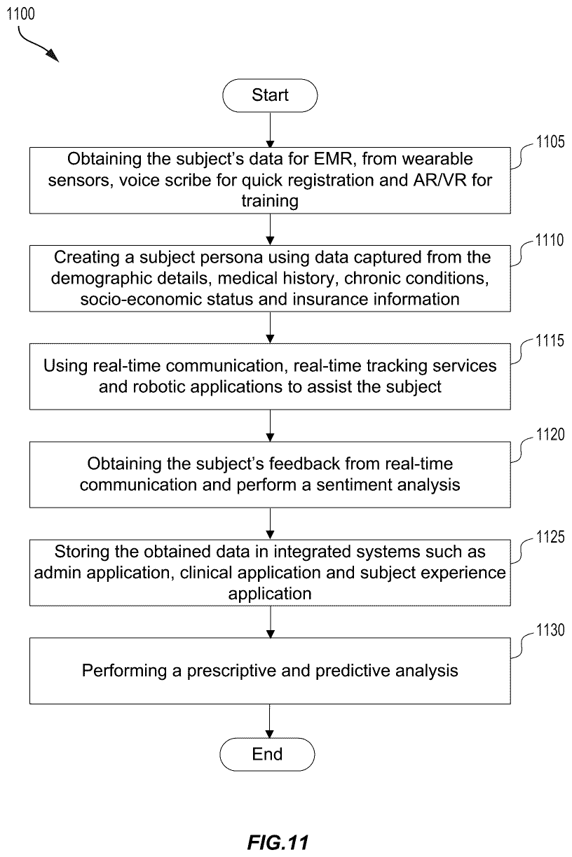

FIG. 11 shows an example flow of data between different modules of a subject centric smart hospital to provide accurate, efficient, and effective care to its subjects. At block 1105, the data of a subject is obtained from EMIR systems of the smart hospital. The data is also collected from different subject-mounted or wearable sensors such as activity trackers, heart rate monitors, sleep trackers, temperature sensors etc. A voice scribe software converts conversations between the medical staff and subjects into a text transcription and then extracts information relevant to a medical record, creates the medical record and stores it into the EMR system of the smart hospital. This process quickly registers a subject that saves times for the stakeholders. At block 1110, a subject persona can be created by using the healthcare data that may have been captured from the demographic details, wearables, medical history, socio-economic status, and insurance billing information. This helps in obtaining collective data of a subject at one place that maintains the integrity of the information of each subject.

At block 1115, a real-time communication link is established between the subject and a health care provider that can enable the real-time engagement between both parties, so that the subject gets accurate, specific and effective treatment. The real-time healthcare tracking and communication technologies can enable timely consultations between healthcare professionals and subjects that can provide healthcare services quickly. To track the vitals, symptoms, and treatment outcomes of subjects using the digital platforms, these services can also enable preemptive interventions and personalized care at block 1115. AI models can assist in these services for a positive outcome for the subjects. Obtaining the feedback of a subject from the above-mentioned process and doing the sentiment analysis at block 1120 to determine positive, negative, or neutral sentiments of subjects can enable health care professionals to address the concerns of subjects and improve the quality of healthcare services and their experience based on the emotional sentiments of subjects. Different applications are used by the subjects as well as by the administration staff for collecting the data as well as for maintaining collaborations. The data obtained from the modules of an integrated system such as administration applications 305, clinical applications 310 and subject applications 315 can be stored in the databases of the cloud platform, so that its security, privacy, and confidentiality can be attained at block 1125. Lastly, the prescriptive and predictive analysis depending on the health status and personas of subjects is performed to reach at a personalized treatment intervention at block 1130 to for each subject.

In some embodiments, a subject may visit a smart hospital offering smart services hosted on a cloud for a routine medical examination. At the reception, the medical and health data of the subject is obtained from the EMR of the hospital by using the secure authentication methods. The subject wears a smartwatch equipped with various sensors to measure the vitals of the subjects that a healthcare provider may use for a scheduled appointment. These sensors monitor the heart rate, blood pressure, and activity levels of a subject in real-time. The data from these subject-mounted devices or wearables is automatically synchronized with the EMR of the hospital to keep the medical record updated with the current health state of a subject. An insurance card can be scanned to provide the information about a corresponding insurance provider for selecting the payment procedures. A voice scribe coupled with a computer vision module may expedite this process. The voice scribe converts the spoken language into text and updates the EMR accordingly, while the computer vision module scans the insurance card, runs OCR on it and extracts relevant information for the billing module and stores it in the EMR of the hospital on the cloud. The automatic collection of health data, wearable sensors data, and registration information can be used to create a personalized subject persona that is specific to the health status of a subject. As the subject proceeds to meet the health provider, the wearable sensors continue to monitor the vitals and the other activities of the subject in real-time. This updated data is also accessible to the healthcare provider in the EMR to save time in measuring it.

In view of the foregoing, the voice scribe can extract the condition of the subject from the conversation with the healthcare provider who can pay personal attention to the subject with empathy as there is no need to take notes or to manually enter the data into the centralized EMR. During the consultation, the subject also shares fears, feelings about how the treatment is going and its impact on its daily life. The sentiment analysis tool detects this sentiment as positive or negative and prompts the provider to adapt its counselling strategy. Once the appointment concludes, the data of subjects including the historical health records, real-time sensor readings, and sentiment feedback, is stored securely in the EMR of the hospital on the cloud platform. The data is gathered from the administration applications 305, clinical application 310, subject applications 315 and several other applications that are used by both the subject and the health care provider. Finally, the health status of a subject is known, and decisions can be made about the treatment plans that are specific to a health profile and/or status of the subject. Several machine learning models may be used to perform automated predictive analysis by reviewing the health status patterns of a subject and subsequently generating alerts. Predictive analysis aids in forecasting potential health hazards that allows a healthcare provider to recommend preventive measures by adopting changes in the lifestyle and diet, and to schedule a personalized follow-up care plan. The seamless data integration, real-time tracking, sentiment analysis, and predictive analysis services operate in an integrated environment of a smart hospital to provide the subject with a personalized evidence-based care that is tailored to the health status and profile of the subject; as a result, the experience of the subject gets positive and the likelihood of enhancing its well-being are significantly enhanced compared to a hospital that provides one-solution-for-one-condition healthcare service.

Real-time tracking of a subject, whether inside or outside a smart hospital facility, can be made possible through a combination of technologies such as wearable devices equipped with GPS, Bluetooth, or RFID technologies. Some of the examples of these devices are smartwatches, badges that can be embedded into clothing or accessories. Inside the hospital, IPS technologies such as Wi-Fi-based triangulation, Geofencing for external areas e.g., parking lots, Bluetooth Low Energy (BLE) beacons, or Ultra-Wideband (UWB) are deployed to accurately track the location of a subject within a facility. A hospital-branded mobile application that subjects install on their smartphones can also be provided to communicate with the wearable devices and the IPS to provide real-time tracking. The real-time data from wearable devices and IPS is transmitted to a central cloud platform or the information system of the hospital. This platform processes and analyzes geolocation data in real-time, detecting anomalies and triggering alerts or notifications. For tracking subjects outside a hospital facility, GPS technology on smartphones may be leveraged. The system may integrate a real-time tracking system with the EMR system of the hospital. This allows healthcare providers to better understand the contextual information of different encounters of the subjects with the healthcare providers.

### EXAMPLES

The following examples illustrate some scenarios in which various aspects of embodiments disclosed herein pertaining to a smart hospital support detections and/or generate insights that would likely be missed by humans

### Example 1; Hereditary Genetic Diseases

A female patient arrives at hospital with her newborn for routine vaccinations. The hospital has a computing system configured such that an artificial intelligence algorithm has access to all electronic medical records in the hospital. The artificial intelligence algorithm detects that the female patient's mother was a carrier of the gene for Hemophilia (a genetic disease). The female patient herself was unaware that her mother had been a carrier for hemophilia. Based on the detection, the artificial intelligence algorithm immediately outputs an alert that recommends that the newborn child be screened for this condition.

Analysis: Without the artificial intelligence algorithm, there would be very little chance that a human doctor would have found out that the patient's mother had been a carrier of hemophilia, since they normally do not open the medical history of the second generation for someone who has come for routine consultation/procedures. This detection by artificial intelligence applies for several genetic diseases such as Sickle Cell Anemia, Tay-Sach's Disease, Cystic Fibrosis, Huntington's Disease, Hereditary Breast and Ovarian Cancer (BRCA1/BRCA2 mutations) etc. This enables early detection, management and saving lives.

### Example 2: Alerting and Preventing Dangerous Food-Drug Interactions

An inpatient in a hospital who had major surgery is on an anti-clotting drug called Warfarin. He had broccoli soup for lunch and plans to have spinach salad and stir-fried chicken for dinner. This meal plan is conveyed to the food department in the hospital. However, an artificial intelligence algorithm detects this meal combination is not compatible with warfarin which the patient is being given. An alert is issued to the department to change his menu for dinner. [Green vegetables have vitamin K which could reduce the anti-clotting ability of Warfarin, causing clots].

Analysis: Without the artificial intelligence algorithm detecting this subtle connection, it would have almost certainly escaped attention of the treating physicians/surgeons.

### Example 3; Pre-Emptively Detecting Probability of Harmful Material Exposure and Alerting

During registration at a hospital, a senior citizen gives her address as Flint, Michigan. She has come with a complaint of constant tiredness and pallor of skin. An artificial intelligence algorithm accesses information from news reports that Flint, Michigan had been known for its high lead content in water. The artificial intelligence algorithm calculates that there is a higher probability that the patient may be having lead poisoning, which could explain her current symptoms. The artificial intelligence algorithm outputs a result to a physician with the physician with the background information and a recommendation to test her serum for lead and hemoglobin levels.

Analysis: Without the artificial intelligence algorithm promptly making the connection between the patient's residential address and her health condition, it would have taken a much longer time, morbidity, expenses and disease progression before the proper diagnosis could be made. This is because typically the residential address of the patient resides in their demographic details, and is not usually considered as a factor in making a diagnosis.

### Example 4: Wearables Based Alert

A patient had a heart attack and bypass surgery was performed. During discharge, the patient is given instructions on when he can get back to normal levels of physical activity. However, the patient got back to his brisk walking routine within 1 week of discharge. An artificial intelligence algorithm in the cloud patient portal (which was monitoring his vitals through the watch) detects that his heart rate is climbing to unsafe levels. Before the rate even reaches the unsafe zone, it issues an alert to both the patient and his primary physician. Notably, the exact duration to refrain from normal physical activity varies between patients, and the artificial intelligence algorithm is configured to assess multiple parameters to understand that 1 week is too little a time for this particular patient to return to his brisk walking routine.

Analysis: In this scenario, the artificial intelligence algorithm is able to pre-emptively sense that the patient is not yet ready for the higher exertion levels that his heart may likely reach if he continues the same levels of activity. It is not possible for a human to continuously monitor this type of vital parameter analysis; moreover, it is complex and tedious for them to do so.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above.

Also, embodiments can be described as a process depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software code can be stored in a memory. Memory can be implemented within the processor or external to the processor. As used herein the term “memory” refers to any type of long term, short term, volatile, nonvolatile, or other storage medium and is not to be limited to any memory or number of memories, or type of media upon which memory is stored.

Moreover, as disclosed herein, the term “storage” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability, or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary details to avoid obscuring the embodiments.

What is claimed is:

1. A computer-implemented method comprising:
collecting data in real-time from one or more data sources for one or more subjects;
aggregating the data of each of the one or more subjects with associated one or more electronic medical records to build a persona for each of the one or more subjects;
deploying at least one service within each sublayer of a subject-centric matrix, wherein each sublayer of a subject centric matrix is customized to include the at least one service for a particular healthcare facility, wherein a service is customized according to the persona associated with each of the one or more subjects, and wherein the service includes a data processing module configured to collect and store data, a communication interface module configured to exchange data, and an artificial intelligence-based engine to perform data analytics on collected data, and wherein the subject-centric matrix includes:
a first sublayer including the one or more first services to know the subject by collecting the data from one or more electronic medical records for creating the persona associated with the subject, or providing a real-time communication between a plurality of subjects;
a second sublayer including the one or more second services for collecting the data from one or more subject-based applications or one or more subject-mounted devices for predictive and prescriptive analysis of the one or more subjects within and outside the healthcare facility; and
a third sublayer including the one or more third services that are based on data-driven and artificial intelligence for enhancing experience of the one or more subjects, the one or more third services include quick registration, augmented/virtual reality (AR/VR), voice scribe, building with internet of things (IoT), or real-time tracking of the one or more subjects; and
deploying a cloud platform configured to host the services, wherein the cloud platform includes:
an interoperability module configured to synchronize a plurality of disparate datasets for establishing data exchange between a plurality of healthcare facilities;
an integration module configured to provide a consistent data format and exchange of the data between sublayers in the subject-centric matrix; and
a customization module configured to customize the one or more services for the particular healthcare facility and for a particular subject according to the persona associated with the subject.

2. The computer-implemented method of claim 1, wherein a data source is a subject-mounted device, a network device, an administration application, a clinical application, or a subject application.

3. The computer-implemented method of claim 1, further comprising:
receiving, via one or more applications and channels, feedback data from the one or more subjects, wherein a feedback is a response to a type of an experience of the one or more subjects for the one or more services;
employing one or more natural language processing (NLP) techniques to preprocess the feedback data;
extracting a subset of the feedback data where the subset represents textual and contextual information;
utilizing topic modeling to categorize the subset of the feedback data into one or more areas wherein each of the one or more areas indicate the type of the experience of the one or more subjects for the one or more services;
utilizing one or more machine learning models to gauge whether the feedback is positive, negative or neutral;
identifying the one or more areas to flag for improvement; and
conducting statistical analysis and data visualization for the one or more subjects to evaluate performance of the one or more services.

4. The computer-implemented method of claim 1, further comprising:
providing security to a real-time communication between one or more devices of a subject and the one or more devices of a healthcare provider by using a secure channel of communication with one or more guardrails to comply with one or more security constraints.

5. The computer-implemented method of claim 1, wherein the one or more third services include virtual reality.

6. The computer-implemented method of claim 1, wherein the one or more third services include augmented reality.

7. A system comprising:
one or more data processors; and
a non-transitory computer readable storage medium containing instructions which, when executed on the one or more data processors, cause the one or more data processors to perform actions including:
collecting data in real-time from one or more data sources for one or more subjects;
aggregating the data of each of the one or more subjects with associated one or more electronic medical records to build a persona for each of the one or more subjects;
deploying at least one service within each sublayer of a subject-centric matrix, wherein each sublayer of a subject centric matrix is customized to include the at least one service for a particular healthcare facility, wherein a service is customized according to the persona associated with each of the one or more subjects, and wherein the service includes a data processing module configured to collect and store data, a communication interface module configured to exchange data, and an artificial intelligence-based engine to perform data analytics on collected data, and wherein the subject-centric matrix includes:
a first sublayer including the one or more first services to know the subject by collecting the data from one or more electronic medical records for creating the persona associated with the subject, or providing a real-time communication between a plurality of subjects;
a second sublayer including the one or more second services for collecting the data from one or more subject-based applications or one or more subject-mounted devices for predictive and prescriptive analysis of the one or more subjects within and outside the healthcare facility; and
a third sublayer including the one or more third services that are based on data-driven and artificial intelligence for enhancing experience of the one or more subjects, the one or more third services include quick registration, augmented/virtual reality (AR/VR), voice scribe, building with internet of things (IoT), or real-time tracking of the one or more subjects; and
deploying a cloud platform configured to host the services, wherein the cloud platform includes:
an interoperability module configured to synchronize a plurality of disparate datasets for establishing data exchange between a plurality of healthcare facilities;
an integration module configured to provide a consistent data format and exchange of the data between sublayers in the subject-centric matrix; and
a customization module configured to customize the one or more services for the particular healthcare facility and for a particular subject according to the persona associated with the subject.

8. The system of claim 7, wherein a data source is a subject-mounted device, a network device, an administration application, a clinical application, or a subject application.

9. The system of claim 7, wherein the actions further include:
receiving, via one or more applications and channels, feedback data from the one or more subjects, wherein a feedback is a response to a type of an experience of the one or more subjects for the one or more services;
employing one or more natural language processing (NLP) techniques to preprocess the feedback data;
extracting a subset of the feedback data where the subset represents textual and contextual information;
utilizing topic modeling to categorize the subset of the feedback data into one or more areas wherein each of the one or more areas indicate the type of the experience of the one or more subjects for the one or more services;
utilizing one or more machine learning models to gauge whether the feedback is positive, negative or neutral;
identifying the one or more areas to flag for improvement; and
conducting statistical analysis and data visualization for the one or more subjects to evaluate performance of the one or more services.

10. The system of claim 7, wherein the actions further include:
providing security to a real-time communication between one or more devices of a subject and the one or more devices of a healthcare provider by using a secure channel of communication with one or more guardrails to comply with one or more security constraints.

11. The system of claim 7, wherein the one or more third services include virtual reality.

12. The system of claim 7, wherein the one or more third services include augmented reality.

13. A computer-program product tangibly embodied in a non-transitory machine-readable storage medium, including instructions configured to cause one or more data processors to perform actions including:
collecting data in real-time from one or more data sources for one or more subjects;
aggregating the data of each of the one or more subjects with associated one or more electronic medical records to build a persona for each of the one or more subjects;
deploying at least one service within each sublayer of a subject-centric matrix, wherein each sublayer of a subject centric matrix is customized to include the at least one service for a particular healthcare facility, wherein a service is customized according to the persona associated with each of the one or more subjects, and wherein the service includes a data processing module configured to collect and store data, a communication interface module configured to exchange data, and an artificial intelligence-based engine to perform data analytics on collected data, and wherein the subject-centric matrix includes:
a first sublayer including the one or more first services to know the subject by collecting the data from one or more electronic medical records for creating the persona associated with the subject, or providing a real-time communication between a plurality of subjects;
a second sublayer including the one or more second services for collecting the data from one or more subject-based applications or one or more subject-mounted devices for predictive and prescriptive analysis of the one or more subjects within and outside the healthcare facility; and
a third sublayer including the one or more third services that are based on data-driven and artificial intelligence for enhancing experience of the one or more subjects, the one or more third services include quick registration, augmented/virtual reality (AR/VR), voice scribe, building with internet of things (IoT), or real-time tracking of the one or more subjects; and
deploying a cloud platform configured to host the services, wherein the cloud platform includes:
an interoperability module configured to synchronize a plurality of disparate datasets for establishing data exchange between a plurality of healthcare facilities;
an integration module configured to provide a consistent data format and exchange of the data between sublayers in the subject-centric matrix; and
a customization module configured to customize the one or more services for the particular healthcare facility and for a particular subject according to the persona associated with the subject.

14. The computer-program product of claim 13, wherein a data source is a subject-mounted device, a network device, an administration application, a clinical application, or a subject application.

15. The computer-program product of claim 13, wherein the actions further include:
receiving, via one or more applications and channels, feedback data from the one or more subjects, wherein a feedback is a response to a type of an experience of the one or more subjects for the one or more services;
employing one or more natural language processing (NLP) techniques to preprocess the feedback data;
extracting a subset of the feedback data where the subset represents textual and contextual information;
utilizing topic modeling to categorize the subset of the feedback data into one or more areas wherein each of the one or more areas indicate the type of the experience of the one or more subjects for the one or more services;
utilizing one or more machine learning models to gauge whether the feedback is positive, negative or neutral;
identifying the one or more areas to flag for improvement; and
conducting statistical analysis and data visualization for the one or more subjects to evaluate performance of the one or more services.

16. The computer-program product of claim 13, wherein the actions further include:
providing security to a real-time communication between one or more devices of a subject and the one or more devices of a healthcare provider by using a secure channel of communication with one or more guardrails to comply with one or more security constraints.

17. The computer-program product of claim 13, wherein the one or more third services include virtual reality.

18. The computer-program product of claim 13, wherein the one or more third services include augmented reality.
